(19) United States
(12) Patent Application Publication
(10) Pub. No.: US 2026/0102609 A1
(43) Pub. Date: Apr. 16, 2026

(54) HIGH FREQUENCY AESTHETIC MEDICAL DEVICE

(71) Applicant: SKINGRAB CO., LTD., Seoul (KR)

(72) Inventor: Sugun LEE, Paju-si (KR)

(73) Assignee: SKINGRAB CO., LTD., Seoul (KR)

(21) Appl. No.: 19/358,163

(22) Filed: Oct. 14, 2025

(30) Foreign Application Priority Data
Oct. 14, 2024 (KR) 10-2024-0139740

(51) Int. Cl. A61N 1/32; A61N 1/06; A61N 1/36; A61N 1/40

(52) U.S. Cl. CPC A61N 1/328; A61N 1/06; A61N 1/36031; A61N 1/403

(57) ABSTRACT

Provided is a high-frequency aesthetic medical device including a needle tip having multiple needle-type electrodes inserted into the skin and formed of conductive and biocompatible metal materials. The device further includes a handpiece coupled to the needle tip, the handpiece having first to third fastening members respectively fastened while the first to third side surfaces of the needle tip are rotated, and a first pin electrically connected to the electrodes. The needle tip includes fourth to sixth fastening members respectively fastened to the first to third fastening members while being rotated, and a connector coupled to the first pin and electrically connected to a second pin connected to the electrodes.

### CROSS-REFERENCE TO RELATED APPLICATIONS

A claim for priority under 35 U.S.C. § 119 is made to Korean Patent Application No. 10-2024-0139740 filed on Oct. 14, 2024 in the Korean Intellectual Property Office, the entire contents of which are hereby incorporated by reference.

### TECHNICAL FIELD

The present disclosure relates to a high frequency aesthetic medical device.

### BACKGROUND

In general, a high frequency aesthetic medical device manages skin by applying high frequency power to the skin to convert electrical energy into bioenergy and promoting fat metabolism and muscle movement through deep heat generated by using the bioenergy.

However, a conventional high frequency aesthetic medical device has low fastening strength between a needle tip and a handpiece, and its usage method is inconvenient.

Furthermore, a conventional high frequency aesthetic medical device cannot efficiently irradiate high frequency energy onto the skin, resulting in low efficiency of skin care and a possibility of burn occurrence.

### PRIOR ART DOCUMENTS

### Patent Document

Korean Registered Patent Publication No. 10-2414073, on Jun. 28, 2022 (Patent Document 1).

### DETAILED DESCRIPTION OF THE INVENTION

### Technical Problem

An embodiment of the present disclosure is directed to providing a device having high fastening strength between a needle tip and a handpiece and a convenient usage method.

An embodiment of the present disclosure is also directed to providing a device capable of efficiently irradiating high frequency energy onto the skin, thereby improving efficiency of skin care.

An embodiment of the present disclosure is also directed to providing a device that includes a sapphire tip and PTFE insulating coated electrodes, and is capable of selectively or simultaneously applying at least one of 6.78 MHz, 13.56 MHz, and 40 MHz and suppressing mutual interference through PLL-based phase synchronization, so that heat may be uniformly distributed to an epidermal layer, a papillary dermal layer, and a reticular dermal layer.

An embodiment of the present disclosure is also directed to providing a device that combines high-speed pumping (2 to 10 Hz) microneedles, impedance feedback of 100 Hz or higher, DCD contact cooling, and RF-synchronized drug spraying, so that hotspot suppression, reduction of energy loss, and enhancement of drug permeability may be achieved.

An embodiment of the present disclosure is also directed to providing a device capable of automatic parameter loading and usage history management through magnetic rotational locking, a multi-pin pogo pin interface, an in-tip memory, and wireless communication.

An embodiment of the present disclosure is also directed to providing a device capable of real-time adjustment of high frequency output based on temperature, resistance, and conductivity of the skin using multiple sensors, and real-time adjustment of high frequency output according to a change value of skin impedance, thereby suppressing occurrence of burns.

### Technical Solution

According to an aspect of the present disclosure, a high frequency aesthetic medical device includes a needle tip that includes a plurality of needle type electrodes inserted into skin, and is manufactured to include a conductive metal and a biocompatible metal material, and a handpiece that is fastened to the needle tip, the handpiece includes a first fastening member that is fastened while a first side surface of the needle tip is rotated, a second fastening member that is fastened while a second side surface of the needle tip is rotated, and including a first pin that is electrically connected to the plurality of electrodes, and a third fastening member that is fastened while a third side surface of the needle tip is rotated, and the needle tip includes a fourth fastening member that is fastened to the first fastening member while being rotated, a fifth fastening member that is fastened to the second fastening member while being rotated, a sixth fastening member that is fastened to the third fastening member while being rotated, and a connector that is coupled to the first pin, and is electrically connected to a second pin electrically connected to the plurality of electrodes.

Furthermore, when the first fastening member and the fourth fastening member, the second fastening member and the fifth fastening member, and the third fastening member and the sixth fastening member reach a first position while being rotated in a first direction, the needle tip and the handpiece may be fastened to each other, and when the first fastening member and the fourth fastening member, the second fastening member and the fifth fastening member, and the third fastening member and the sixth fastening member reach an initial position from the first position while being rotated in a second direction, the needle tip and the handpiece may be unfastened from each other.

Furthermore, the first fastening member may include a first protrusion hole, the fourth fastening member may include a fourth protruding projection that is inserted into the first protrusion hole, and is fastened to the first protrusion hole while being rotated, the second fastening member may include a second fastening groove, the fifth fastening member may include a fifth fastening projection that is inserted into the second fastening groove, and is fastened to the second fastening groove while being rotated, the third fastening member may include a third fastening hole, and the sixth fastening member may include a sixth fastening projection that is inserted into the third fastening hole, and that is fastened to the third fastening hole while being rotated.

Furthermore, the first pin may be configured to be surrounded by the first fastening member and the third fastening member, and the second pin may be configured to intensively apply a high frequency signal in response to a level of high frequency energy received through the first pin coupled to the connector, to a target portion of the skin through the plurality of electrodes.

Furthermore, the second pin may be configured to selectively apply, among the high frequency signals, high frequency signals having different frequency bands to a specific layer of the skin through the plurality of electrodes, or simultaneously apply high frequency signals having different frequency bands to different layers of the skin.

Furthermore, the needle tip may include a first printed circuit board, on which a first memory is mounted, the first memory may be configured to store at least one data, among temperature data, impedance data, and shot count data of high frequency irradiation acquired by the sensor module, and the high frequency aesthetic medical device further may include a cable that is electrically connected to the first printed circuit board and a second printed circuit board such that the at least one data stored in the first memory is stored in a second memory mounted on the second printed circuit board of the handpiece.

Furthermore, a first wireless communication module may be further mounted on the first printed circuit board, a second wireless communication module may be further mounted on a second printed circuit board of the handpiece, and at least one data stored in the first memory may be stored in a second memory mounted on the second printed circuit board through wireless communication, through the first wireless communication module and the second wireless communication module.

Furthermore, the first wireless communication module may transmit first usage history information stored in the first memory to the handpiece or the server, and the second wireless communication module may transmit second usage history information stored in the second memory to the needle tip or the server.

Furthermore, the needle tip may include a sensor module that acquires a temperature, a resistance, and a conductivity of the skin, and a second control module that is provided on a second printed circuit board of the handpiece outputs a target high frequency signal preset in association with a temperature, a resistance, and a conductivity of the skin, based on the acquired temperature, resistance, and conductivity of the skin.

Furthermore, the handpiece may include a touchscreen panel that is configured such that an output form of a high frequency signal set to irradiate a high frequency to the skin, a form of a frequency band, and a treatment time are changed through a user interface.

Furthermore, the handpiece may be configured to be fastened to the needle tip based on a non-contact magnetic coupling.

Furthermore, a fastening strength of the needle tip and the handpiece may be automatically adjusted depending on a condition of the skin.

Furthermore, the needle tip may be provided with single-crystal sapphire, and the needle tip may include a PTFE insulating coating layer that is formed on an outer surface of the needle tip and that applies high frequency energy to the skin through a capacitive coupling scheme between a plurality of electrodes and the skin.

Furthermore, the handpiece may include a feedback module that measures an impedance of the skin by using a sampling frequency, and compensates a high frequency output such that a measured impedance value is maintained within a preset target impedance range.

Furthermore, the handpiece may include a contact cooling module that cools an epidermis by spraying dynamic cooling device (DCD) gas before or after a high frequency is irradiated to the skin.

Furthermore, the handpiece may include a drug delivery module that sprays a drug to the skin in synchronization with an irradiation of the high frequency.

Furthermore, a high frequency generating module provided in the second printed circuit board may include a phase synchronization circuit that selectively or simultaneously outputs at least one high frequency signal, and maintains a phase error between the output high frequency signals at a preset level or less.

Furthermore, the phase synchronization circuit may simultaneously apply a plurality of high frequency signals or perform sequential switching based on a direct digital synthesis (DDS) scheme.

Furthermore, the high frequency energy may be selected as a single frequency or as multiple frequencies, and a processor may perform a control such that an output, a phase, and a frequency of the high frequency energy are set mutually differently.

According to an aspect of the present disclosure, a method for controlling a high frequency aesthetic medical device includes preliminarily cooling skin by performing contact cooling on the skin through a needle tip, by a processor of the high frequency aesthetic medical device, monitoring an impedance of the skin in real time while a plurality of electrodes are driven at a preset period to be moved forward to a target depth, by the processor, phase-synchronizing at least one high frequency energy through a phase synchronization circuit and applying the high frequency energy, by the processor, measuring the impedance by using a sampling frequency, and automatically adjusting a high frequency output in response to a measured impedance value, by the processor, and spraying a drug to the skin through a drug delivery module such that the spraying is synchronized with application of a high frequency energy, by the processor.

### BRIEF DESCRIPTION OF DRAWINGS

FIGS. 1 to 9 are views illustrating a high frequency aesthetic medical device according to the present disclosure.

FIGS. 10 to 14 are views illustrating a high frequency irradiating process of a high frequency aesthetic medical device according to the present disclosure.

FIG. 15 is a view illustrating a state, in which a needle tip and a handpiece of the present disclosure are electrically connected to each other.

FIG. 16 is a view illustrating a method for controlling a high frequency aesthetic medical device according to the present disclosure.

FIG. 17 is a view illustrating a configuration of a handpiece of the present disclosure as another example.

FIG. 18 is a view illustrating an operation principle of a composite electrode structure including a needle tip provided with single-crystal sapphire and a PTFE insulating coating layer, as an example.

FIG. 19 is a view illustrating a process of synthesizing a composite frequency signal and controlling an output signal through a high frequency generating module of the present disclosure, as an example.

FIG. 20 is a view illustrating a magnetic rotational locking and multi-pin interface structure of the present disclosure, as an example.

### MODE FOR CARRYING OUT THE INVENTION

Throughout this specification, when it is supposed that a member is located on another member “on”, this includes not only the case where one member is in contact with another member but also the case where another member is present between two other members.

Terms such as ‘first’, ‘second’, and the like are used to distinguish one component from another component, and thus the component is not limited by the terms described above. Unless there are obvious exceptions in the context, a singular form includes a plural form.

Hereinafter, an operation principle and embodiments of the present disclosure will be described with reference to the accompanying drawings.

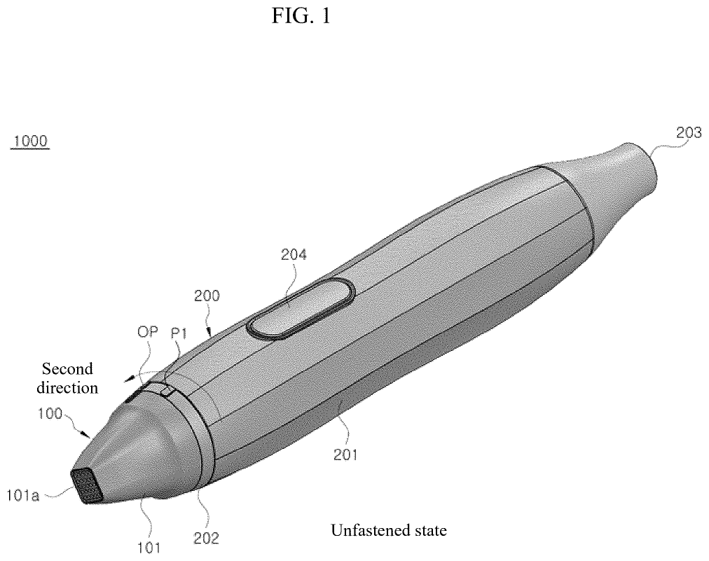

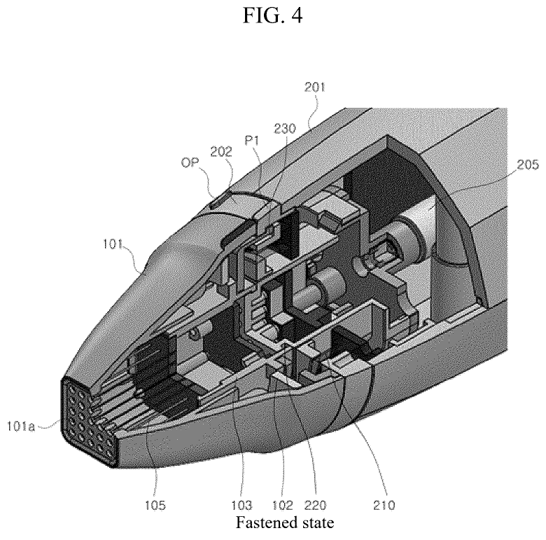

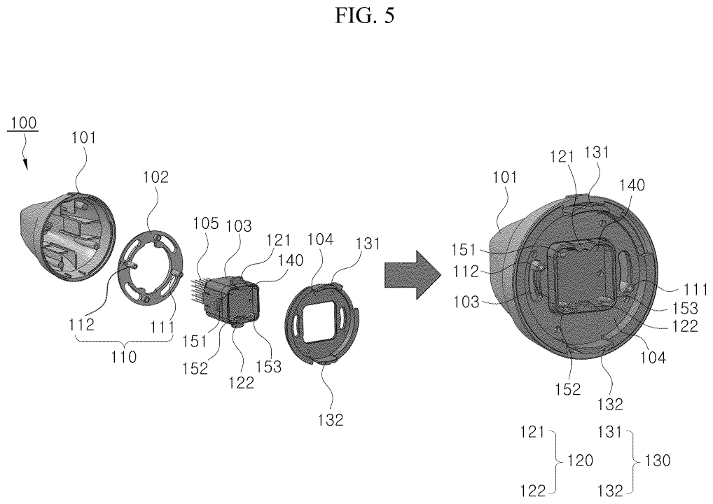

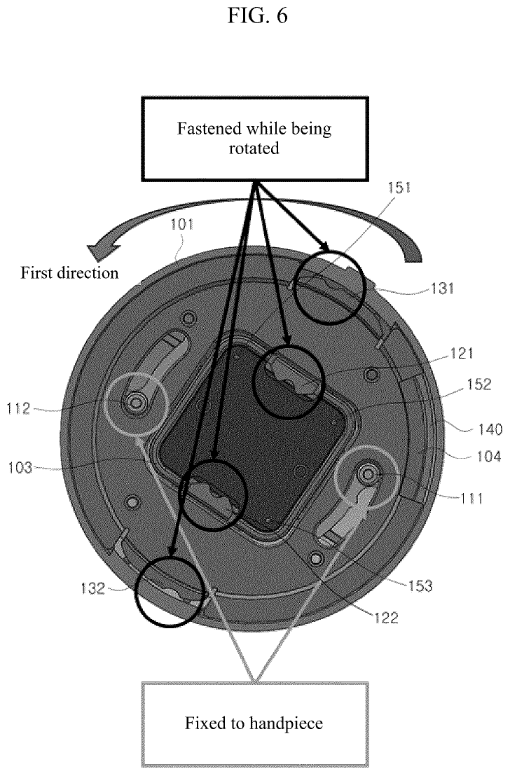

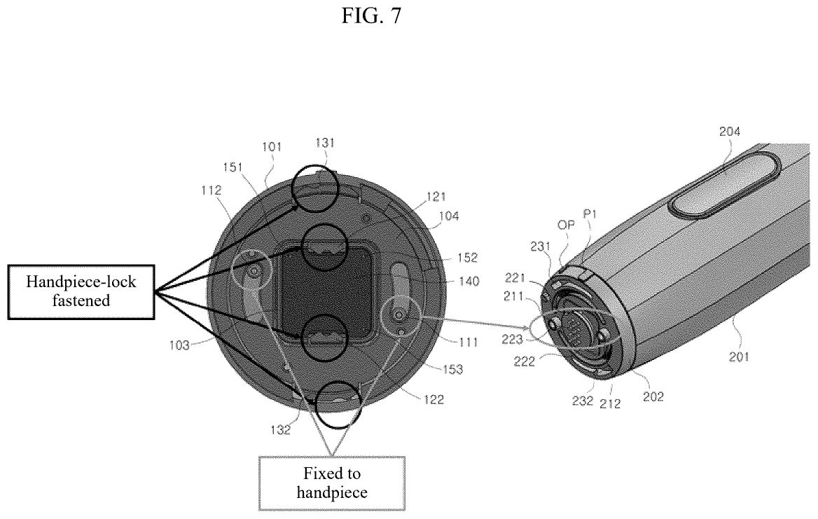

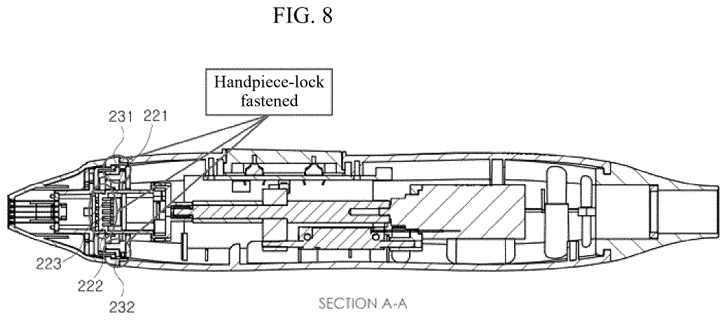

FIGS. 1 to 9 are views illustrating a high frequency aesthetic medical device according to the present disclosure.

As illustrated in FIGS. 1 to 9, a high frequency aesthetic medical device 1000 may include a needle tip 100 and a handpiece 200.

The needle tip 100 may include a plurality of needle-type electrodes 105 that are inserted into skin. The needle tip 100 may be manufactured to include a conductive metal and a biocompatible metal material. The conductive metal may be silver (Ag) having a high electrical conductivity, or aluminum (Al) having the highest electrical conductivity in consideration of cost efficiency. The biocompatible metal material may be at least one of stainless steel, cobalt-based alloy, and titanium-based alloy that are non-toxic, non-carcinogenic, and resistant to a corrosive environment in a human body, and may be at least one of gold and amalgam that are widely used for dental materials. Such a needle tip 100 may reduce stimulation of the skin and optimize electrical conductivity.

The handpiece 200 may be configured to be fastened to the needle tip 100 based on a non-contact magnetic coupling. The handpiece 200 may include a body 201, a first cover 202, a second cover 203, a button 204, a first fastening member 210, a second fastening member 220, and a third fastening member 230.

The first body 201 may be configured to be fastened to the first cover 202 and the second cover 203. The button 204 may turn on or off irradiation of high frequency through a pressing operation.

The first fastening member 210 may be provided in the first cover 202, and may be configured to be fastened based on a non-contact magnetic coupling while a first side surface of the needle tip 100 is rotated. At least one of the first cover 202 and the first fastening member 210 may be manufactured to include a magnetic material, and may include a plurality of first protrusion holes 211 and 212.

The second fastening member 220 may be provided in the first cover 202, may be configured to be fastened based on a non-contact magnetic coupling while a second side surface of the needle tip 100 is rotated, and may include a plurality of first pins 223 that are configured to be electrically connected to a plurality of electrodes 105. The second fastening member 220 may be manufactured to include a magnetic material, and may include a plurality of second fastening grooves 221 and 222.

The third fastening member 230 may be provided in the first cover 202, and may be configured to be fastened based on a non-contact magnetic coupling while a third side surface of the needle tip 100 is rotated. The third fastening member 230 may be manufactured to include a magnetic material, and may include a plurality of third fastening holes 231 and 232.

The needle tip 100 may include a first case 101, a plate 102, a housing 103, a second case 104, a fourth fastening member 110, a fifth fastening member 120, and a sixth fastening member 130.

The housing 103 may be configured to be fastened by the first case 101, the plate 102, and the second case 104. The first case 101 may include a plurality of electrode holes 101a, through which the plurality of electrodes 105 pass. At least one of the first case 101, the plate 102, the housing 103, and the second case 104 may be manufactured to include a magnetic material.

The fourth fastening member 110 may be configured to be fastened to the first fastening member 210 based on a non-contact magnetic coupling while being rotated. The fourth fastening member 110 may include a plurality of fourth protruding projections 111 and 112 that are inserted into the plurality of first protrusion holes 211 and 212, and are fastened to the plurality of first protrusion holes 211 and 212 based on a non-contact magnetic coupling while being rotated. The fourth fastening member 110 may be manufactured to include a magnetic material.

The fifth fastening member 120 may be configured to be fastened to the second fastening member 220 based on a non-contact magnetic coupling while being rotated. The fifth fastening member 120 may include a plurality of fifth fastening projections 121 and 122 that are inserted into the plurality of second fastening grooves 221 and 222, and are fastened to the plurality of second fastening grooves 221 and 222 based on a non-contact magnetic coupling while being rotated. The fifth fastening member 120 may be manufactured to include a magnetic material.

The sixth fastening member 130 may be configured to be fastened to the third fastening member 230 based on a non-contact magnetic coupling while being rotated. The sixth fastening member 130 may include a plurality of sixth fastening projections 131 and 132 that are inserted into the plurality of third fastening holes 231 and 232, and are fastened to the plurality of third fastening holes 231 and 232 based on a non-contact magnetic coupling while being rotated. The sixth fastening member 130 may be manufactured to include a magnetic material.

In the present disclosure, in the high frequency aesthetic medical device 1000, when the first fastening member 210 and the fourth fastening member 110, the second fastening member 220 and the fifth fastening member 120, and the third fastening member 230 and the sixth fastening member 130 reach a first position P1 while being rotated in the first direction, the needle tip 100 and the handpiece 200 may be fastened to each other based on a non-contact magnetic coupling.

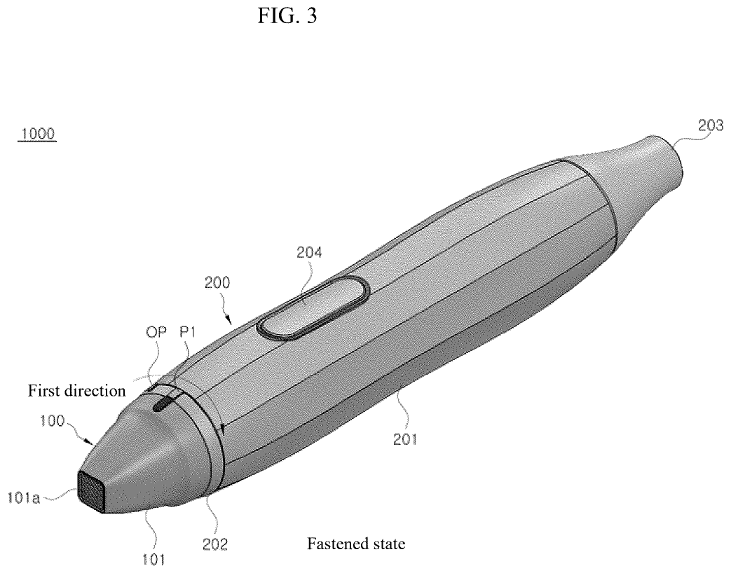

That is, as illustrated in FIGS. 3 to 8, in the high frequency aesthetic medical device 1000, when a plurality of fourth protruding projections 111 and 112 that are fastened to the plurality of first protrusion holes 211 and 212 based on a non-contact magnetic coupling while being rotated, a plurality of fifth fastening projections 121 and 122 that are fastened to the plurality of second fastening grooves 221 and 222 based on a non-contact magnetic coupling while being rotated, and a plurality of sixth fastening projections 131 and 132 that are fastened to the plurality of third fastening holes 231 and 232 based on a non-contact magnetic coupling while being rotated reach the first position P1 while being rotated in the first direction, the needle tip 100 and the handpiece 200 may be fastened to each other based on a non-contact magnetic coupling. An actuator 205 may move the plurality of electrodes 105 such that the plurality of electrodes 105 reach a target depth in the skin. This may increase user convenience and reduce a fastening error.

In the present disclosure, a fastening strength of the needle tip 100 and the handpiece 200 of the high frequency aesthetic medical device 1000 may be automatically adjusted depending on a condition of the skin. That is, the fastening strength of the needle tip 100 and the handpiece 200 may be automatically adjusted based on a non-contact magnetic coupling depending on a roughness condition of the skin when a high frequency is irradiated. Furthermore, the fastening strength of the needle tip 100 and the handpiece 200 may be automatically adjusted based on a non-contact magnetic coupling depending on an uneven portion and an angled portion of the face. The present disclosure enables customized fastening for each user through automatic adjustment of the fastening strength.

In the present disclosure, in the high frequency aesthetic medical device 1000, when the first fastening member 210 and the fourth fastening member 110, the second fastening member 220 and the fifth fastening member 120, and the third fastening member 230 and the sixth fastening member 130 reach an initial position OP from a first position P1 while being rotated in a second direction, the needle tip 100 and the handpiece 200 may be unfastened from each other.

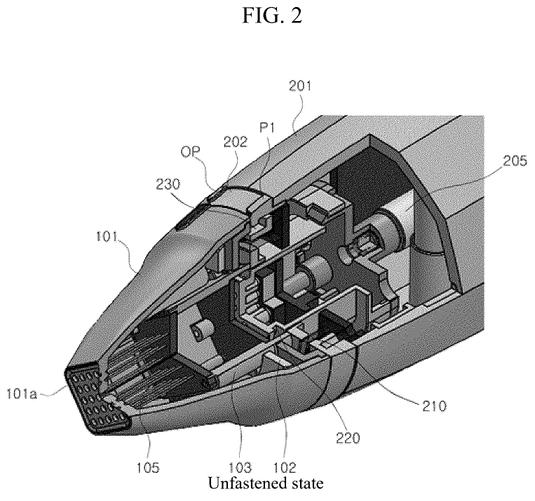

That is, as illustrated in FIGS. 1 and 2, in the high frequency aesthetic medical device 1000, when a plurality of fourth protruding projections 111 and 112 that are fastened to the plurality of first protrusion holes 211 and 212 based on a non-contact magnetic coupling, a plurality of fifth fastening projections 121 and 122 that are fastened to the plurality of second fastening grooves 221 and 222 based on a non-contact magnetic coupling, and a plurality of sixth fastening projections 131 and 132 that are fastened to the plurality of third fastening holes 231 and 232 based on a non-contact magnetic coupling reach the initial position OP from the first position P1 while being rotated in the second direction, the needle tip 100 and the handpiece 200 may be unfastened from each other.

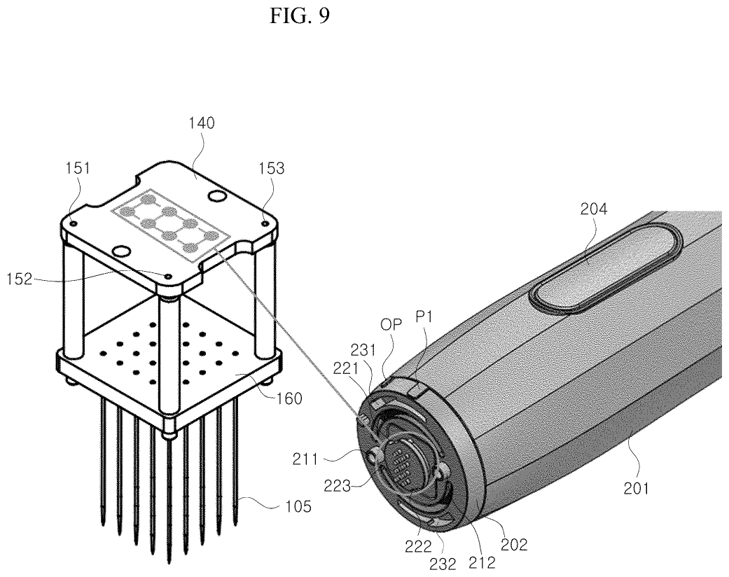

As illustrated in FIG. 9, a connector 140 may be coupled to the plurality of first pins 223, and may be configured to be electrically connected to a plurality of second pins 151, 152, and 153 that are electrically connected to the plurality of electrodes 105 mounted on a first printed circuit board 160. The plurality of first pins 223 may be configured to be surrounded by the first fastening member 210 and the third fastening member 230. The connector 140 may be a pogo pin connector, the plurality of second pins 151, 152, and 153 may be a plurality of pogo pins, and the number of the plurality of pogo pins is not limited.

Furthermore, the plurality of second pins 151, 152, and 153 may be configured to selectively apply high frequency signals having different frequency bands, among high frequency signals according to levels of high frequency energy received through the plurality of first pins 223 coupled to the connector 140, to a specific layer of the skin through the plurality of electrodes 105, or to simultaneously apply the high frequency signals having different frequency bands to different layers of the skin. The present disclosure may be configured such that a low frequency signal according to a level of low frequency energy may be received through a first pin 223, and a low frequency signal and a high frequency signal may be simultaneously applied to a deep layer, such as an epidermal layer of the skin, through the plurality of electrodes 105. For example, the deep layer may be any one of a dermal layer, a subcutaneous fat layer, and an SMAS layer.

The plurality of second pins 151, 152, and 153 may be configured to intensively apply a high frequency signal according to a level of high frequency energy received through the plurality of first pins 223 coupled to the connector 140, to a target area of the skin, through the plurality of electrodes 105. In the present disclosure, because a concentration degree of energy between the electrodes may be increased, an efficiency of radiation of high frequency may be improved.

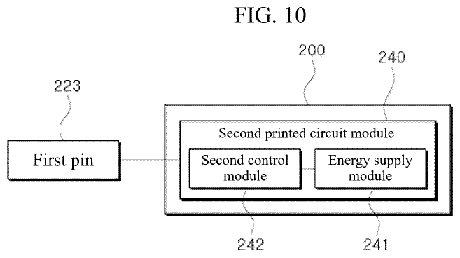

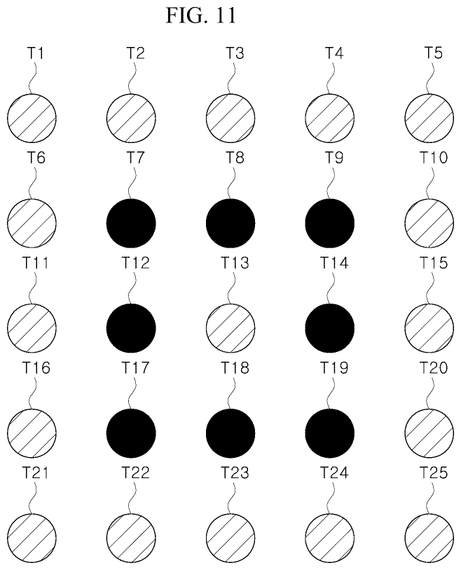

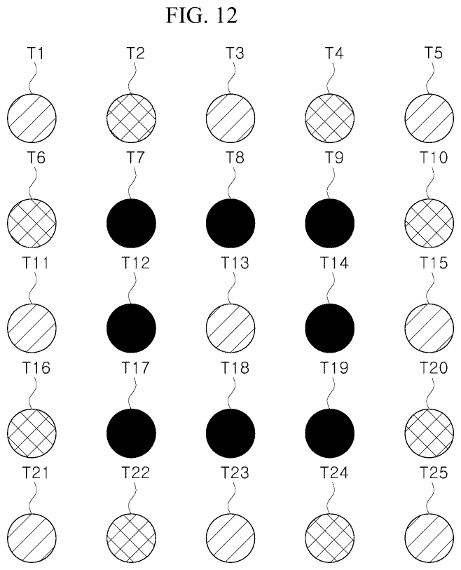

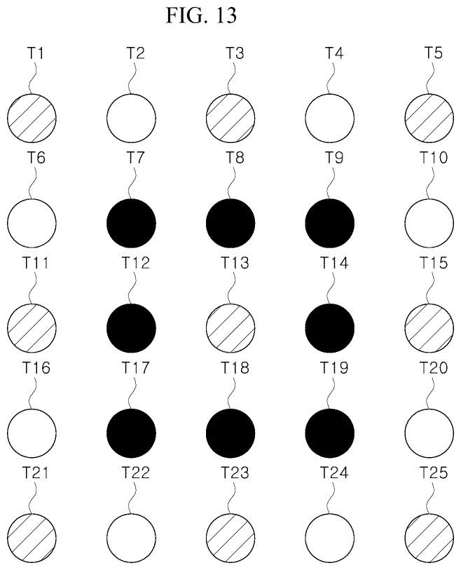

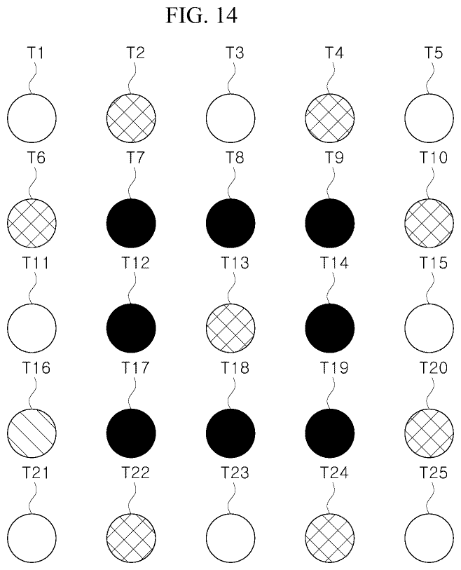

FIGS. 10 to 14 are views illustrating a high frequency irradiating process of a high frequency aesthetic medical device according to the present disclosure.

As illustrated in FIGS. 9 and 10, the plurality of second pins 151 and 153 may be configured to apply a first high frequency signal and a second high frequency signal, received through the plurality of first pins 223 coupled to the connector 140, to the plurality of electrodes 105. The plurality of first pins 223 may be electrically connected to a second printed circuit board 240 of the handpiece 200.

A second control module 242 may be provided in the second printed circuit board 240 of the handpiece 200, and when the plurality of electrodes 105 reach a target depth, an energy supply module 241 may be controlled such that the first high frequency signal and the second high frequency signal are applied to the plurality of electrodes 105. The energy supply module 241 may include a switching circuit for differently applying the first high frequency signal and the second high frequency signal.

As illustrated in FIGS. 9 to 11, the second control module 242 may control the energy supply module 241 such that, when the plurality of electrodes 105 reach a target depth, with a specific shot of irradiation of high frequency, 1 MHz energy corresponding to the first high frequency signal is applied to all positions T1 to T5 of a first row having a (+) polarity electrically connected to a second pin 151, the first position T6 and the fifth position T10 of a second row, the first position T11, the third position T13, and the fifth position T15 of a third row, the first position T16 and the fifth position T20 of a fourth row, and all positions T21 to T25 of a fifth row.

The second control module 242 may control the energy supply module 241 such that, when the plurality of electrodes 105 reach a target depth, with a cross shot of irradiation of high frequency, 1 MHz energy corresponding to the second high frequency signal is applied to all positions T1 to T5 of a first row having a (+) polarity electrically connected to the second pin 151, the first position T6 and the fifth position T10 of a second row, the first position T11, the third position T13, and the fifth position T15 of a third row, the first position T16 and the fifth position T20 of a fourth row, and all positions T21 to T25 of a fifth row.

Thereafter, the second control module 242 may control the energy supply module 241 such that 2 MHz energy corresponding to the second high frequency signal is cross-applied to all positions T1 to T5 of a first row having a (+) polarity electrically connected to the second pin 151, the first position T6 and the fifth position T10 of a second row, the first position T11, the third position T13, and the fifth position T15 of a third row, the first position T16 and the fifth position T20 of a fourth row, and all positions T21 to T25 of a fifth row.

Furthermore, the second position T7, the third position T8, and the fourth position T9 of a second row, the second position T12 and the fourth position T14 of a third row, and the second position T17, the third position T18, and the fourth position T19 of a fourth row may be positions having a (−) polarity electrically connected to the second pin 153.

As illustrated in FIGS. 9 and 10, the plurality of second pins 151, 152, and 153 may be configured to apply a third high frequency signal, received through the plurality of first pins 223 coupled to the connector 140, to the plurality of electrodes 105. The plurality of first pins 223 may be electrically connected to the second printed circuit board 240 of the handpiece 200.

The second control module 242 may be provided in the second printed circuit board 240 of the handpiece 200, and when the plurality of electrodes 105 reach a target depth, the energy supply module 241 may be controlled such that the third high frequency signal is applied to the plurality of electrodes 105. The energy supply module 241 may include a switching circuit for differently applying the third high frequency signal.

As illustrated in FIGS. 9, 10, and 12, the second control module 242 may control the energy supply module 241 such that, when the plurality of electrodes 105 reach a target depth, with a cross shot of irradiation of high frequency, 1 MHz energy corresponding to the third high frequency signal is applied to the first position T1, the third position T3, and the fifth position T5 of a first row having a (+) polarity electrically connected to the second pin 151, the first position T11, the third position T13, and the fifth position T15 of a third row, and the first position T21, the third position T23, and the fifth position T25 of a fifth row, and 2 MHz energy corresponding to the third high frequency signal is applied to the second position T2, the fourth position T4, and the sixth position T6 of the first row, the second position T12, the fourth position T14, and the sixth position T16 of the third row, and the first position T21, the third position T23, and the fifth position T25 of the fifth row.

Thereafter, the second control module 242 may control the energy supply module 241 such that 2 MHz energy corresponding to the third high frequency signal is cross-applied to the second position T2 and the fourth position T4 of a first row, the first position T6 and the fifth position T10 of a second row, the first position T16 and the fifth position T20 of a fourth row, and the second position T22 and the fourth position T24 of a fifth row, each having a (+) polarity electrically connected to the second pin 152.

Furthermore, the second position T7, the third position T8, and the fourth position T9 of a second row, the second position T12 and the fourth position T14 of a third row, and the second position T17, the third position T18, and the fourth position T19 of a fourth row may be positions having a (−) polarity electrically connected to the second pin 153.

As illustrated in FIGS. 9 and 10, the plurality of second pins 151 and 153 may be configured to apply a fourth high frequency signal and a fifth high frequency signal, received through the plurality of first pins 223 coupled to the connector 140, to the plurality of electrodes 105. The plurality of first pins 223 may be electrically connected to the second printed circuit board 240 of the handpiece 200.

The second control module 242 may be provided in the second printed circuit board 240 of the handpiece 200, and when the plurality of electrodes 105 reach a target depth, the energy supply module 241 may be controlled such that the fourth high frequency signal and the fifth high frequency signal are applied to the plurality of electrodes 105. The energy supply module 241 may include a switching circuit for differently applying the fourth high frequency signal and the fifth high frequency signal.

As illustrated in FIGS. 9, 10, and 13, the second control module 242 may control the energy supply module 241 such that, when the plurality of electrodes 105 reach a target depth, with a specific shot of irradiation of high frequency, 1 MHz energy corresponding to the fourth high frequency signal is applied to the first position T1, the third position T3, and the fifth position T5 of a first row having a (+) polarity electrically connected to the second pin 151, the first position T11, the third position T13, and the fifth position T15 of a third row, and the first position T21, the third position T23, and the fifth position T25 of a fifth row.

The second control module 242 may control the energy supply module 241 such that, when the plurality of electrodes 105 reach a target depth, with a cross shot of irradiation of high frequency, 1 MHz energy corresponding to the fifth high frequency signal is applied to the first position T1, the third position T3, and the fifth position T5 of a first row having a (+) polarity electrically connected to the second pin 151, the first position T11, the third position T13, and the fifth position T15 of a third row, and the first position T21, the third position T23, and the fifth position T25 of a fifth row.

Thereafter, the second control module 242 may control the energy supply module 241 such that 2 MHz energy corresponding to the fifth high frequency signal is cross-applied to the first position T1, the third position T3, and the fifth position T5 of a first row having a (+) polarity electrically connected to the second pin 151, the first position T11, the third position T13, and the fifth position T15 of a third row, and the first position T21, the third position T23, and the fifth position T25 of a fifth row.

Furthermore, the second position T7, the third position T8, and the fourth position T9 of a second row, the second position T12 and the fourth position T14 of a third row, and the second position T17, the third position T18, and the fourth position T19 of a fourth row may be positions having a (−) polarity electrically connected to the second pin 153.

Furthermore, the second control module 242 may control the energy supply module 241 such that 1 MHz energy corresponding to the fourth high frequency signal, and 1 MHz energy and 2 MHz energy corresponding to the fifth high frequency signal, are not applied to the second position T2 and the fourth position T4 of a first row, the first position T6 and the fifth position T10 of a second row, the first position T16 and the fifth position T20 of a fourth row, and the second position T22 and the fourth position T24 of a fifth row.

As illustrated in FIGS. 9 and 10, the plurality of second pins 152 and 153 may be configured to apply a sixth high frequency signal and a seventh high frequency signal, received through the plurality of first pins 223 coupled to the connector 140, to the plurality of electrodes 105. The plurality of first pins 223 may be electrically connected to the second printed circuit board 240 of the handpiece 200.

The second control module 242 may be provided in the second printed circuit board 240 of the handpiece 200, and when the plurality of electrodes 105 reach a target depth, the energy supply module 241 may be controlled such that the sixth high frequency signal and the seventh high frequency signal are applied to the plurality of electrodes 105. The energy supply module 241 may include a switching circuit for differently applying the sixth high frequency signal and the seventh high frequency signal.

As illustrated in FIGS. 9, 10, and 14, the second control module 242 may control the energy supply module 241 such that, when the plurality of electrodes 105 reach a target depth, with a specific shot of irradiation of high frequency, 1 MHz energy corresponding to the sixth high frequency signal is applied to the second position T2 and the fourth position T4 of a first row having a (+) polarity electrically connected to the second pin 152, the first position T6 and the fifth position T10 of a second row, the third position T13 of a third row, the first position T16 and the fifth position T20 of a fourth row, and the second position T22 and the fourth position T24 of a fifth row.

The second control module 242 may control the energy supply module 241 such that, when the plurality of electrodes 105 reach a target depth, with a cross shot of irradiation of high frequency, 1 MHz energy corresponding to the seventh high frequency signal is applied to the second position T2 and the fourth position T4 of a first row having a (+) polarity electrically connected to the second pin 152, the first position T6 and the fifth position T10 of a second row, the third position T13 of a third row, the first position T16 and the fifth position T20 of a fourth row, and the second position T22 and the fourth position T24 of a fifth row.

Thereafter, the second control module 242 may control the energy supply module 241 such that 2 MHz energy corresponding to the seventh high frequency signal is cross-applied to the second position T2 and the fourth position T4 of a first row, the first position T6 and the fifth position T10 of a second row, the third position T13 of a third row, the first position T16 and the fifth position T20 of a fourth row, and the second position T22 and the fourth position T24 of a fifth row, each having a (+) polarity electrically connected to the second pin 152.

Furthermore, the second position T7, the third position T8, and the fourth position T9 of a second row, the second position T12 and the fourth position T14 of a third row, and the second position T17, the third position T18, and the fourth position T19 of a fourth row may be positions having a (−) polarity electrically connected to the second pin 153.

Furthermore, the second control module 242 may control the energy supply module 241 such that 1 MHz energy corresponding to the sixth high frequency signal, and 1 MHz energy and 2 MHz energy corresponding to the seventh high frequency signal, are not applied to the first position T1, the third position T3, and the fifth position T5 of a first row, the first position T11 and the fifth position T15 of a third row, and the first position T21, the third position T23, and the fifth position T25 of a fifth row.

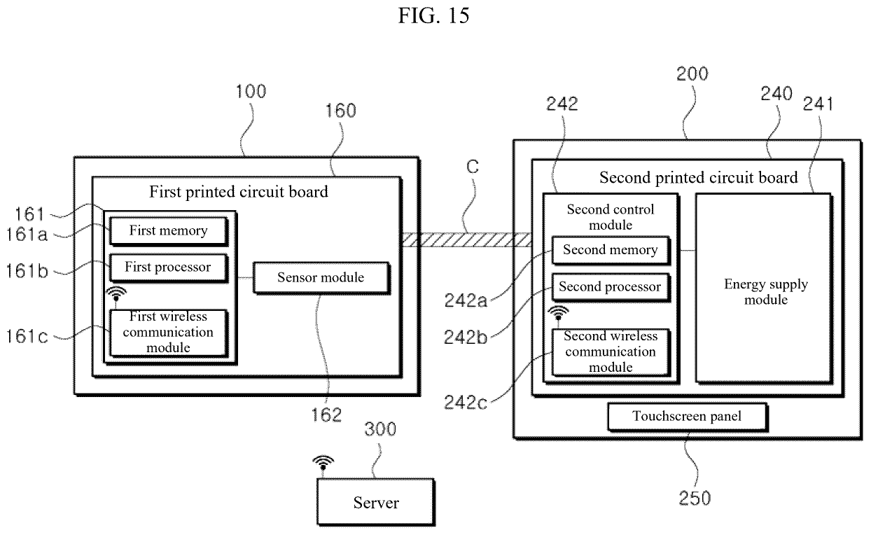

FIG. 15 is a view illustrating a state, in which a needle tip and a handpiece of the present disclosure are electrically connected to each other.

As illustrated in FIG. 15, the cable C may be electrically connected to the first printed circuit board 160 and the second printed circuit board 240.

The needle tip 100 may include a first printed circuit board 160, on which a first control module 161 and a sensor module 162 are mounted. The first control module 161 may include a first memory 161a and a first processor 161b. The first memory 161a may store at least one data, among temperature data, impedance data, and shot count data of irradiation of high frequency, which are acquired by the sensor module 162. The first processor 161b may transmit at least one data, among temperature data, impedance data, and shot count data of irradiation of high frequency, to the second control module 242 of the handpiece 200.

The second control module 242 may include a second memory 242a and a second processor 242b. The second processor 242b may store at least one data, among temperature data, impedance data, and shot count data of irradiation of high frequency, which are received from the first processor 161b, in the second memory 242a. The second processor 242b may control the energy supply module 241 such that high frequency energy is irradiated as preset target energy for respective positions of the plurality of electrodes 150, based on at least one data among temperature data, impedance data, and shot count data of irradiation of high frequency.

The sensor module 162 may acquire a temperature, a resistance, and a conductivity of the skin. The sensor module 162 may acquire a change value of impedance of the skin. The sensor module 162 may include multiple sensors. The multiple sensors may acquire a temperature, a resistance, and a conductivity of the skin while monitoring them in real time, and may acquire a change value of impedance of the skin while monitoring it in real time.

The second control module 242 may output a preset target high frequency signal in association with a temperature, a resistance, and a conductivity of the skin, based on the acquired temperature, resistance, and conductivity of the skin. The second control module 242 may adjust a high frequency output according to a temperature, a resistance, and a conductivity of the skin in real time, based on the target high frequency signal. In the present disclosure, because a high frequency output may be adjusted in real time depending on a temperature, a resistance, and a conductivity of the skin, a burn of the skin may be prevented.

The second control module 242 may output a preset target high frequency signal in association with a change value of impedance of the skin, based on the acquired change value of impedance of the skin. The second control module 242 may adjust a high frequency output according to a change value of impedance of the skin in real time, based on the target high frequency signal. In the present disclosure, because a high frequency output may be adjusted in real time depending on a change value of impedance of the skin, a burn of the skin may be prevented.

The handpiece 200 may include a touchscreen panel 250 to change an output form of a high frequency signal, a form of a frequency band, and a treatment time set for irradiating high frequency to the skin through a user interface. The user may adjust an operation of irradiating high frequency while changing an output form of a high frequency signal, a form of a frequency band, and a treatment time in real time by using a smart handle including the touchscreen panel 250. The present disclosure may improve user convenience, and may prevent a burn of the skin.

A first wireless communication module 161c may transmit at least one data stored in the first memory 161a to a second wireless communication module 242c. The second memory 242a may store at least one data stored in the first memory 161a through the second wireless communication module 242c. In the present disclosure, data may be efficiently stored in a memory through wireless communication.

The first wireless communication module 161c may transmit first usage history information stored in the first memory 161a to the handpiece 200 or a server 300. The second wireless communication module 242c may transmit second usage history information stored in the second memory 242a to the needle tip 100 or the server 300. The first usage history information and the second usage history information may be usage history data including user-specific customized treatment data according to irradiation of high frequency. In the present disclosure, because data may be shared with each other through wireless communication, inconvenience of cable connection may be reduced, and because user-specific customized treatment data may be transmitted to the server 300, usage statistics may be collected through data linkage with the server 300, and the user-specific customized treatment data may be efficiently utilized based on the usage statistics.

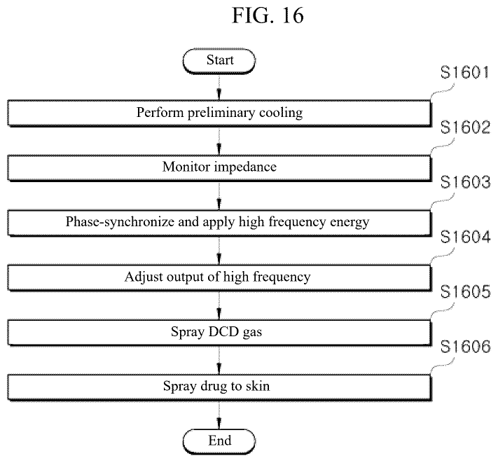

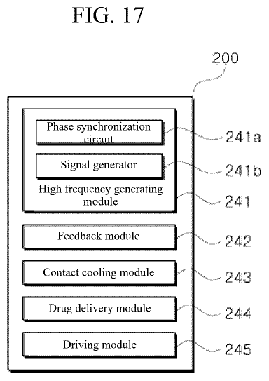

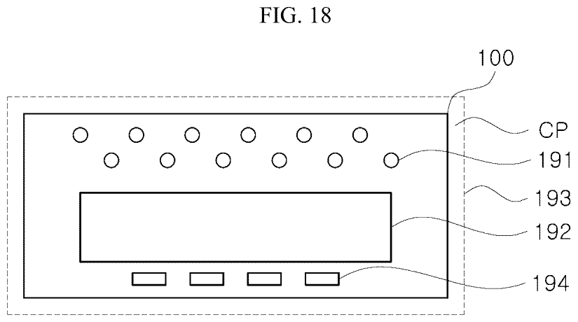

FIG. 16 is a view illustrating a method for controlling a high frequency aesthetic medical device according to the present disclosure. FIG. 17 is a view illustrating a configuration of a handpiece of the present disclosure as another example. FIG. 18 is a view illustrating an operation principle of a composite electrode structure including a needle tip provided with single-crystal sapphire and a PTFE insulating coating layer according to the present disclosure, as an example.

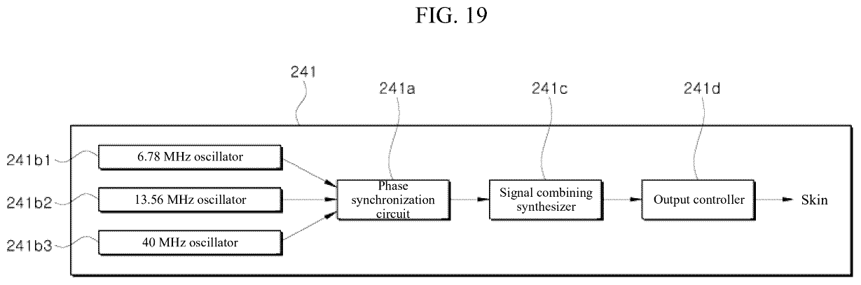

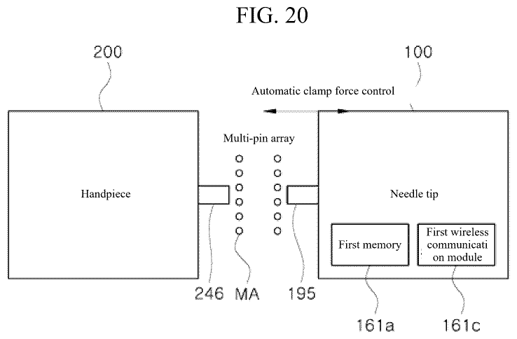

FIG. 19 is a view illustrating a process of synthesizing a composite frequency signal and controlling an output signal through a high frequency generating module according to the present disclosure, as an example. FIG. 20 is a view illustrating a magnetic rotational locking and multi-pin interface structure of the present disclosure, as an example.

As illustrated in FIGS. 16 to 20, a method for controlling the high frequency aesthetic medical device 1000 according to the present disclosure may include a first operation S1601, a second operation S1602, a third operation S1603, a fourth operation S1604, a fifth operation S1605, and a sixth operation S1606.

The second processor 242b according to the present disclosure may be configured to perform preliminary cooling by performing contact cooling on skin through the needle tip 100 provided with single-crystal sapphire (S1601).

In this case, the high frequency aesthetic medical device 1000 may include a needle tip 100 provided with single-crystal sapphire that contacts the skin, a plurality of electrodes 105 that are formed on an outer surface of the needle tip 100, and a polytetrafluoroethylene (PTFE) insulating coating layer 193 that covers the plurality of electrodes 105.

As an example, a surface roughness Ra of the needle tip 100 provided with single-crystal sapphire may be within a range of 0.1 μm to 0.3 μm, or may be formed in a nano pattern having a period of 100 nm to 500 nm. The surface characteristics of the needle tip 100 provided with single-crystal sapphire may improve durability and functionality.

Furthermore, a thickness of the PTFE insulating coating layer 193 may be in a range of 10 μm to 50 μm, and a dielectric constant thereof may be 2.0 to 2.2. In this case, the PTFE insulating coating layer 193 may be formed in the thickness and dielectric constant ranges to secure insulation characteristics and durability, and through this, electrical insulation performance may be effectively exerted.

Here, high frequency energy may be configured to be delivered between the plurality of electrodes 105 and the skin in a capacitive coupling scheme, and through this, high frequency energy may be effectively applied to the skin. In this case, the plurality of electrodes 105 may be configured to perform a high-speed high frequency applying operation after a low-speed low frequency applying operation is performed, by a pumping mechanism that is driven by a frequency of 2 Hz to 10 Hz.

The first processor 161b according to the present disclosure may be configured to monitor an impedance of the skin in real time while moving the plurality of electrodes 105 arranged in a microneedle array to a target depth at a period of 2 Hz to 5 Hz (S1602). The first processor 161b according to the present disclosure may be configured to phase-synchronize and apply at least one of 6.78 MHz, 13.56 MHz, and 40 MHz energy through a high frequency generating module 241, and to perform a pumping operation at a period of 7 Hz to 10 Hz (S1603).

Here, the high frequency generating module 241 may be provided in the second printed circuit board 240 included in the handpiece 200, and the high frequency generating module 241 may be configured to selectively or simultaneously generate and output a high frequency signal having at least one of 6.78 MHz, 13.56 MHz, and 40 MHz frequencies.

In this case, the high frequency generating module 241 may include a phase adjusting circuit 241a that controls a phase error between the corresponding high frequency signals to be maintained at ±1° or less when the high frequency signals are output simultaneously. Through this, even when multiple frequencies are applied, interference between signals may be minimized, and stable energy delivery or stimulation effects may be implemented.

Furthermore, the high frequency generating module 241 may include a function of simultaneously applying a plurality of high frequency signals or sequentially switching the high frequency signals based on a direct digital synthesis (DDS) scheme.

The first processor 161b may be configured to secure efficiency and safety of energy delivery by measuring skin impedance at a sampling frequency of 100 Hz or higher through a feedback module 242 and automatically adjusting a high frequency output according to the measured impedance value (S1604).

Here, the handpiece 200 may include a feedback module 242, and the feedback module 242 may be configured to measure an impedance of the skin in real time at a sampling frequency of 100 Hz or higher. In this case, the feedback module 242 may control a high frequency output to automatically adjust or compensate for an intensity or waveform of the output, so that the measured impedance value is maintained within a preset target impedance range. Through this, automatic optimization of output conditions according to changes in skin conditions may be achieved, and skin safety of the user and precision of treatment effects may be improved.

In detail, the first processor 161b may perform a control to primarily lower the plurality of electrodes 105 arranged in a microneedle array form to a target depth first, and sequentially apply high frequency energy to a plurality of intermediate target depth points while raising the plurality of electrodes 105 arranged in the microneedle array form.

That is, the first processor 161b may be configured such that the plurality of electrodes 105 arranged in the microneedle array form may reciprocate to and from the skin. In this case, the first processor 161b may primarily lower the plurality of electrodes 105 arranged in the microneedle array form to a lowest target depth, and in the following raising process, may perform a control to sequentially apply high frequency energy at a plurality of intermediate target depths, so that the high frequency energy may be efficiently delivered to different depths of the skin. Through this, the first processor 161b may maximize a treatment effect and prevent excessive thermal damage to the epidermal layer or the dermal layer by performing energy application that is differentiated for respective depths of multilayered skin tissues.

In this case, the target depths are mapped in a depth table including preset thicknesses of skin, pain reaction data, and reference data according to treated portions, and the first processor 161b may update the depth table in response to bio-signals measured based on the depth table. Through this, a treatment depth may be dynamically adjusted according to bio-characteristics of each patient.

Here, the first processor 161b may interlock position sensor signals of the plurality of electrodes 105 arranged in a microneedle array form and control signals of the high frequency generating module 241 to efficiently deliver energy for respective target depths. The first processor 161b may optimize a distribution of energy in a treatment portion by adjusting an output and an application time of high frequency energy whenever respective intermediate target depths are reached. In this case, sequential application of high frequency energy may enable an effective treatment while minimizing damage to skin tissues.

The first processor 161b may perform a control to apply high frequency energy again while secondarily lowering the plurality of electrodes 105 arranged in a microneedle array form from a target depth after allowing them to re-enter, and raising the plurality of electrodes 105 arranged in the microneedle array form.

That is, the first processor 161b may be configured to control the plurality of electrodes 105 arranged in a microneedle array form to temporarily stop at a target depth at a level of a lower end of the epidermis before they completely deviate from a surface of the skin, and to apply high frequency energy in a process of the plurality of electrodes 105 being raised again after re-entering from the target depth and performing a second lowering operation. Through the control scheme, the first processor 161b may precisely adjust entry and rising paths of the plurality of electrodes 105 arranged in the microneedle array form, and may apply high frequency energy step by step to a necessary depth, so that a distribution of thermal stimulation on a treatment portion may be optimized and a delivery efficiency of the high frequency energy may be improved.

In this case, the first processor 161b may more precisely adjust a distribution of energy in the skin tissue through a second lowering operation and re-application of the high frequency energy to increase efficiency and safety of the treatment.

Here, for a target depth, a re-entry speed, and output parameters of high frequency energy, at least one of impedance, temperature, and contact force of the skin may be measured by the first processor 161b in real time with a sampling frequency of 100 Hz or more, and a control signal for correcting the target depth, a re-entry speed profile, and a magnitude and a pulse width of the high frequency energy applied at a plurality of intermediate target depths in a closed-loop control scheme based on the measured data may be generated. Through this, in correspondence to variation factors that occur during a treatment, delivery of high frequency energy may be optimized, and treatment effects and safety may be improved.

In this case, an initiation condition for re-entry may be defined as a case, in which impedance changes by a preset specific ratio or more as compared with a reference value, or a change amount of contact force per unit time is equal to or greater than a preset reference value. Through this, a change in skin condition may be sensed in real time, and a re-entry operation may be efficiently controlled in response thereto.

The first processor 161b may sense a change in the skin condition by analyzing biometric signal data collected at high speed, and may dynamically adjust an entry depth and a movement speed of the plurality of electrodes 105 arranged in the microneedle array form, and an output intensity and a pulse width of the high frequency energy, and a parameter of a frequency. Through this, by coping promptly with changes in skin reactions that may occur during a treatment, an optimum treatment effect may be maintained while excessive thermal damage or pain is prevented.

The first processor 161b may perform a control to apply energy at a precise depth in a closed-loop scheme by linking position information of the plurality of electrodes 105 arranged in the microneedle array form and an output control signal of the high frequency generating module 241 in real time. In this case, because the closed-loop control is automatically performed in response to a bio-signal that is fed back in real time, a precise control may be achieved without separate intervention of an operator, and a customized treatment according to individual skin characteristics of a patient may be enabled.

The first processor 161b may control such that a second application depth set is selected to be identical to or different from a first application depth set and is applied. Furthermore, the first processor 161b may be configured to select and re-irradiate only some of the depths included in the first application depth set when the second application depth set is set, or to include an additional depth that does not overlap the first application depth set. In this case, the first processor 161b may perform a control to maximize treatment efficiency and minimize side effects in a closed-loop scheme by analyzing data collected during a treatment and dynamically updating the second application depth set. Through this, a customized treatment according to individual skin characteristics of a patient may be enabled by differently setting the first application depth set and the second application depth set.

The first processor 161b may perform a control to apply high frequency energy only in a process of repeating a treatment two or more times on the same treatment portion and raising the plurality of electrodes 105 arranged in the microneedle array form. Furthermore, the first processor 161b may perform a control to optimize a cumulative thermal distribution by differently setting at least one of output, pulse width, and frequency of the high frequency energy in a first pass and a second pass. Through this, a delivery efficiency of the high frequency energy may be increased, and a treatment effect may be maximized.

Specifically, the first processor 161b may precisely control a distribution of thermal energy delivered to tissues of a treatment portion by differently setting an output intensity, a pulse width, a frequency, and a phase of the high frequency energy for each signal delivery pass. Through this, tissue damage caused by excessive thermal concentration may be prevented, while uniform and effective thermal treatment for a target depth and range may be enabled.

Furthermore, the first processor 161b may independently control high frequency output conditions for each pass, in conjunction with a high frequency generating module 241 that includes a signal generator 241b and a phase control circuit 241a corresponding to each pass, so as to minimize signal interference between a plurality of passes and optimize a distribution of the high frequency energy. Through such multi-pass control, treatment efficiency may be improved, and side effects may be reduced.

Furthermore, the first processor 161b may differently adjust parameters of each pass depending on skin characteristics and a treatment purpose, and may be configured to achieve safer and more precise treatment effects by dynamically optimizing an output condition of high frequency energy applied at each pass based on skin condition and impedance data measured in real time during a treatment.

The first processor 161b may precisely control heat transfer to a treatment portion by performing a control to repeatedly perform, at least two times, a series of procedures including a stop operation of temporarily stopping after the plurality of electrodes 105 arranged in the microneedle array form reach a level of a lower end of the epidermis, a lowering operation of performing a second lowering, and a re-application operation of re-applying the high frequency energy.

In this case, the first processor 161b may be configured to make a spatial distribution of thermal energy in the skin tissue uniform by applying high frequency energy step by step while the plurality of electrodes 105 arranged in the microneedle array form perform a plurality of lowering and rising operations in the vicinity of a target depth through repeated control, and to maximize a treatment effect through this.

Furthermore, the first processor 161b may be configured to simultaneously secure safety and treatment efficiency of a treatment operation by dynamically adjusting a lowering depth, a stop time, and an application intensity and duration of high frequency energy for each operation, based on position information of the plurality of electrodes 105 arranged in the microneedle array form and skin condition data collected in real time during a repeated control process.

Furthermore, the first processor 161b may be configured to individually set repeated control conditions according to an anatomical structure of a treatment region, a skin thickness, an impedance variation, and a response characteristic of a patient, so that a customized treatment protocol may be implemented, and excessive thermal accumulation or tissue damage during the treatment may be prevented.

The first processor 161b may perform a control to include a pause section between an operation in which the plurality of electrodes 105 arranged in the microneedle array form stop at a level of a lower end of the epidermis and an operation in which a second lowering is performed.

That is, the first processor 161b may perform a control to set a pause section between a first lowering and raising operation and a second lowering and raising operation, and may selectively control such that a depth set of high frequency energy applied during the second lowering operation is identical to or different from a depth set applied during the first lowering operation. Through this, the first processor 161b may be configured to flexibly adjust a depth at which high frequency energy is applied according to at least one of a treatment condition, a skin condition, and a treatment purpose, so that precision of energy distribution within a treatment region may be improved and a more effective and customized treatment may be enabled.

In this case, the pause section may have a time range of 50 ms to 500 ms. The pause section may provide time for the skin tissue to stabilize in response to thermal stimulation caused by the high frequency energy applied in a preceding operation, so that overheating of the skin tissue may be prevented and damage to the treatment region may be minimized.

Furthermore, the first processor 161b may be configured to drive a cooling signal or a soothing signal during the pause section to alleviate thermal stimulation of the skin surface and tissue, so that safety of the treatment and comfort of the patient may be improved.

Furthermore, the first processor 161b may be configured to variably adjust a time of the pause section according to a skin condition of a patient, characteristics of a treatment region, and application conditions of high frequency energy, so that interference between repeated lowering and raising operations may be minimized, an optimum treatment effect may be induced, and side effects may be reduced.

The first processor 161b according to the present disclosure may be configured to spray DCD gas for 30 ms to 100 ms at a pre-treatment time point and for 50 ms to 150 ms at a post-treatment time point (S1605).

Here, the handpiece 200 may include a contact cooling module 243, and the contact cooling module 243 may be configured to cool an epidermis by spraying DCD gas for 30 ms to 150 ms according to a timing before or after high frequency irradiation is performed. Here, spraying of the DCD gas may be performed for the purpose of reducing thermal damage of the epidermis that may be caused by the high frequency energy, alleviating pain during the treatment, and enhancing a skin protection effect for the user. In this case, a timing and a duration of a spraying sequence may be controlled by the second control module 242 depending on a treatment condition or a skin condition.

Furthermore, the first processor 161b may be controlled to additionally perform an operation of providing at least one of cooling by spraying DCD gas, surface anesthesia, and a soothing signal, during the pause section. In this case, the first processor 161b may spray DCD gas onto a skin surface of a treatment portion during a duration of the pause section to induce a local temperature drop, thereby effectively alleviating thermal stimulation caused by application of high frequency energy, and at the same time, may apply a surface anesthetic or output a soothing signal in parallel to reduce pain and discomfort of a patient. Such combined cooling and soothing actions may prevent skin damage that may occur during or immediately after the treatment, and may shorten a recovery time of the patient.

Furthermore, the first processor 161b may be configured to implement a user-customized treatment environment by selecting at least one of cooling, surface anesthesia, and soothing means or by driving a combination of a plurality of means, based on collected data corresponding to a treatment condition, a skin temperature, an impedance variation, and a skin sensitivity of a patient.

The first processor 161b according to the present disclosure may be configured to spray a drug onto the skin in synchronization with application of high frequency energy (S1606).

In this case, the handpiece 200 may include a drug delivery module 244, and the drug delivery module 244 may be configured to spray a drug onto the skin in synchronization with a timing of applying high frequency energy. Here, the drug delivery module 244 may be controlled such that drug spraying is performed during or immediately after application of the high frequency energy to the skin, so that permeability of the drug may be improved by utilizing temporarily changed permeability characteristics of the skin caused by the high frequency energy. In this case, at least one of a type of the drug to be sprayed, a spray amount, and a spray pressure may be configured to be adjustable depending on a skin condition or a treatment purpose.

Furthermore, the first processor 161b may be configured to stably stop at a level of a lower end of the epidermis during a treatment by controlling, based on a sensor signal for sensing positions of the plurality of electrodes 105 arranged in the microneedle array form in real time, such that the plurality of electrodes 105 arranged in the microneedle array form are not excessively exposed to the outside of the skin. Through this, the first processor 161b may prevent the plurality of electrodes 105 arranged in the microneedle array form from completely deviating from the skin tissue, and may minimize pain and skin damage during a treatment through stable position control at a level of a lower end of the epidermis.

Furthermore, the first processor 161b may, for the same treatment region, perform a second lowering operation by temporarily stopping the plurality of electrodes 105 arranged in the microneedle array form at a target depth of a lower end of the epidermis and re-entering them, and perform a control to apply high frequency energy, so that the treatment region may be repeatedly stimulated and the high frequency energy may be precisely delivered. In this case, the target depth may be selected as a depth within an upper range of a papillary dermis from a lower end of an epidermis, and the first processor 161b may perform a control such that the target depth includes a safety limit depth capable of preventing complete discharge to the outside of the skin, whereby safety may be secured such that the plurality of electrodes 105 arranged in the microneedle array form do not excessively penetrate the skin.

The first processor 161b may precisely control the plurality of electrodes 105 arranged in the microneedle array form not to be completely discharged to the outside of the skin during a treatment, so that a treatment effect may be maximized through repeated micro-stimulation and application of high frequency energy, and pain and discomfort of a patient may be effectively reduced.

Meanwhile, the first processor 161b according to the present disclosure may estimate an absolute depth of the plurality of electrodes 105 arranged in the microneedle array form by using data of at least one sensor, among a motor encoder and a linear scale received from a driving module 245 provided in the handpiece 200, and may control such that the estimated absolute depth does not increase to a preset minimum deviation reference or more, so that the plurality of electrodes 105 arranged in the microneedle array form may perform a software-based blocking function for preventing complete exposure to the outside of the skin. In this case, the first processor 161b may perform a blocking function in a real-time feedback control scheme in conjunction with the driving module 245, and through this, may prevent skin damage and user discomfort caused by excessive penetration of the plurality of electrodes 105 arranged in the microneedle array form, and may secure safety during a treatment.

The first processor 161b according to the present disclosure may perform a control to ensure that the needle tip 100 and the handpiece 200 are firmly connected to each other based on a magnetic rotational locking fastening structure and a connector 140. In this case, the connector 140 may be provided as a pogo pin connector.

Furthermore, the first processor 161b may perform a control to store, in a first memory 161a, usage history data of at least one of usage parameters, temperature, impedance, number of shots, and indication-related information, and may be configured to transmit the stored data to an external device through a first wireless communication module 161c. Through this, a usage state and history of the needle tip 100 may be managed in real time, and efficient control based on treatment history may be enabled.

Furthermore, the first processor 161b may perform a control to automatically adjust a fastening strength of the magnetic rotational locking fastening structure based on physical characteristics or condition information of the skin, so that stable fastening force according to a usage environment and user convenience may be simultaneously secured.

In addition, the first processor 161b may be configured to analyze data stored in the first memory 161a and detected state information, set optimum fastening conditions and energy application conditions suitable for treatment conditions, and perform linked control through wireless communication with an external system, so that safety of an entire system and precision of treatment may be improved.

As illustrated in FIG. 18, an operation principle of a composite electrode structure including a needle tip provided with single-crystal sapphire and a PTFE insulating coating layer is as follows.

First, the second control module 242 may spray DCD gas for 30 ms to 100 ms through a spiral cooling channel 191 to pre-cool a skin surface.

Subsequently, an inner high frequency electrode 192 may receive a high frequency signal of at least one of 6.78 MHz, 13.56 MHz, or 40 MHz from a high frequency generating module 241, and may deliver the signal to the plurality of electrodes 105. Here, the plurality of electrodes 105 may apply high frequency energy to the skin in a capacitive coupling (CP) scheme through a PTFE insulating coating layer 193.

In this case, an impedance sensor 194 may measure impedance of the skin in real time with a sampling frequency of 100 Hz or more, and the second control module 242 may automatically adjust high frequency output according to the measured impedance value. Through this, to promptly cope with changes in skin condition, excessive thermal damage may be prevented, and an optimum treatment effect may be maintained.

Thereafter, the second control module 242 may spray DCD gas for 50 ms to 150 ms through the spiral cooling channel 191 after irradiation of high frequency energy is completed, so that the epidermis may be cooled and thermal damage may be prevented.

In the present disclosure, a high thermal conductivity and a low contact resistance of the needle tip 100 provided with single-crystal sapphire, a capacitive coupling characteristic of the PTFE insulating coating layer 193, a uniform cooling effect of the spiral cooling channel 191, and real-time feedback control of the impedance sensor 194 are combined, so that spatial uniformity of energy distribution may be improved, hotspot generation may be suppressed, and output loss due to reduction of contact resistance may be decreased.

As illustrated in FIG. 19, the high frequency generating module 241 may include a plurality of oscillators 241b1, 241b2, and 241b3 for simultaneously or sequentially generating high frequency signals of different frequency bands, a phase synchronization circuit 241a for precisely correcting phase differences between the plurality of oscillators 241b1, 241b2, and 241b3, a signal combining synthesizer 241c that combines phase-synchronized signals to synthesize them into a single or composite frequency signal, and an output controller 241d that controls an output signal.

In this case, the plurality of oscillators 241b1, 241b2, and 241b3 may generate high frequency signals having frequency bands of approximately 6.78 MHz, 13.56 MHz, and 40 MHz, respectively, and the phase synchronization circuit 241a may perform a control such that a phase difference (inter-frequency phase error) between frequency signals that are output from the plurality of oscillators 241b1, 241b2, and 241b3 is ±1° or less.

Furthermore, the signal combining synthesizer 241c may generate a high frequency signal having a desired frequency combination by combining a plurality of frequency components or selectively synthesizing them in a sequential switching scheme based on direct digital synthesis (DDS).

Furthermore, the output controller 241d may control output power, phase, and frequency sequence of a generated high frequency signal, and finally apply the output signal to skin of a treatment target.

In the present disclosure, by applying a plurality of high frequencies in a mutually phase-synchronized state, energy distribution according to impedance characteristics of each skin layer may be optimized, mutual interference between frequencies may be minimized, and a stable and uniform therapeutic high frequency output may be provided.

As illustrated in FIG. 20, the magnetic rotational locking and multi-pin interface structure may include the handpiece 200 and the needle tip 100. Here, the handpiece 200 and the needle tip 100 may be mechanically coupled to each other through a magnetic coupling member 195 and a rotational locking member 246. In this case, the rotational locking member 246 may be fastened or released according to an operation of being rotated at a specific angle, and the magnetic coupling member 195 may include a magnetic material or a pair of magnets, so that the handpiece 200 and the needle tip 100 may be magnetically coupled in an aligned state.

Furthermore, the handpiece 200 may be formed with a multi-pin array (MA) for transmission and reception of electrical signals, and the needle tip 100 may be provided with a contact structure that is designed to be paired with the multi-pin array (MA), so that power, high frequency signals, and control signals may be electrically connected.

In this case, in the magnetic rotational locking and multi-pin interface structure, automatic clamping force control may be performed by a magnetic flux or mechanical position sensor during fastening, so that when the needle tip 100 is attached to the handpiece 200, a fastening strength may be automatically adjusted.

Furthermore, because the needle tip 100 includes a first memory 161a for storing at least data of at least one of usage history, number of shots, temperature, and impedance, and a first wireless communication module 161c for performing wireless communication with the handpiece 200 or an external device, easy assembly and disassembly through magnetic fastening may be achieved, stability of signal transmission may be secured through the multi-pin electrical connection structure, and user operation convenience and fastening reliability may be improved through automatic clamping force control.

According to the above-described means for solving the problems of the present disclosure, high fastening strength between the needle tip and the handpiece may be achieved, and a convenient usage method may be provided.

In addition, according to the above-described means for solving the problems of the present disclosure, spatial uniformity of energy distribution may be improved, output loss due to reduction of contact resistance may be decreased, and hotspot generation may be suppressed through a combination of thermal conductivity and contact resistance characteristics of sapphire, capacitive coupling based on dielectric properties of PTFE, and multi-frequency phase-synchronized operation.

In addition, according to the above-described means for solving the problems of the present disclosure, a treatment time may be shortened through layer-by-layer parallel stimulation, and a synergistic effect of enhancing drug delivery efficiency may be provided through high-speed pumping and RF-synchronized drug spraying.

In addition, according to the above-described means for solving the problems of the present disclosure, high frequency energy may be efficiently irradiated onto the skin, thereby improving efficiency of skin care.

In addition, according to the above-described means for solving the problems of the present disclosure, high frequency output may be real-time adjusted based on temperature, resistance, and conductivity of the skin using multiple sensors, and may also be real-time adjusted according to a change value of skin impedance, thereby suppressing occurrence of burns.

As described above, the embodiments of the present disclosure are exemplary and should not be construed as limiting.

1. A high frequency aesthetic medical device comprising:
a needle tip including a plurality of needle type electrodes inserted into skin, and manufactured to include a conductive metal and a biocompatible metal material; and
a handpiece configured to be fastened to the needle tip,
wherein the handpiece includes:
a first fastening member configured to be fastened while a first side surface of the needle tip is rotated;
a second fastening member configured to be fastened while a second side surface of the needle tip is rotated, and including a first pin configured to be electrically connected to the plurality of electrodes; and
a third fastening member configured to be fastened while a third side surface of the needle tip is rotated, and
wherein the needle tip includes:
a fourth fastening member configured to be fastened to the first fastening member while being rotated;
a fifth fastening member configured to be fastened to the second fastening member while being rotated;
a sixth fastening member configured to be fastened to the third fastening member while being rotated; and
a connector coupled to the first pin, and electrically connected to a second pin electrically connected to the plurality of electrodes.

2. The high frequency aesthetic medical device of claim 1, wherein when the first fastening member and the fourth fastening member, the second fastening member and the fifth fastening member, and the third fastening member and the sixth fastening member reach a first position while being rotated in a first direction, the needle tip and the handpiece are fastened to each other, and
wherein when the first fastening member and the fourth fastening member, the second fastening member and the fifth fastening member, and the third fastening member and the sixth fastening member reach an initial position from the first position while being rotated in a second direction, the needle tip and the handpiece are unfastened from each other.

3. The high frequency aesthetic medical device of claim 2, wherein the first fastening member includes a first protrusion hole,
wherein the fourth fastening member includes a fourth protruding projection inserted into the first protrusion hole, and fastened to the first protrusion hole while being rotated,
wherein the second fastening member includes a second fastening groove,
wherein the fifth fastening member includes a fifth fastening projection inserted into the second fastening groove, and fastened to the second fastening groove while being rotated,
wherein the third fastening member includes a third fastening hole, and
wherein the sixth fastening member includes a sixth fastening projection inserted into the third fastening hole, and fastened to the third fastening hole while being rotated.

4. The high frequency aesthetic medical device of claim 3, wherein the first pin is configured to be surrounded by the first fastening member and the third fastening member, and wherein the second pin is configured to intensively apply a high frequency signal in response to a level of high frequency energy received through the first pin coupled to the connector, to a target portion of the skin through the plurality of electrodes.

5. The high frequency aesthetic medical device of claim 4, wherein the second pin is configured to selectively apply, among the high frequency signals, high frequency signals having different frequency bands to a specific layer of the skin through the plurality of electrodes, or simultaneously apply high frequency signals having different frequency bands to different layers of the skin.

6. The high frequency aesthetic medical device of claim 1, wherein the needle tip includes a first printed circuit board, on which a first memory is mounted,
wherein the first memory is configured to store at least one data, among temperature data, impedance data, and shot count data of high frequency irradiation acquired by the sensor module, and
wherein the high frequency aesthetic medical device further includes:
a cable electrically connected to the first printed circuit board and a second printed circuit board such that the at least one data stored in the first memory is stored in a second memory mounted on the second printed circuit board of the handpiece.

7. The high frequency aesthetic medical device of claim 6, wherein a first wireless communication module is further mounted on the first printed circuit board, wherein a second wireless communication module is further mounted on a second printed circuit board of the handpiece, and
wherein at least one data stored in the first memory is stored in a second memory mounted on the second printed circuit board through wireless communication, through the first wireless communication module and the second wireless communication module.

8. The high frequency aesthetic medical device of claim 7, wherein the first wireless communication module transmits first usage history information stored in the first memory to the handpiece or the server, and
wherein the second wireless communication module transmits second usage history information stored in the second memory to the needle tip or the server.

9. The high frequency aesthetic medical device of claim 1, wherein the needle tip includes:
a sensor module configured to acquire a temperature, a resistance, and a conductivity of the skin, and
wherein a second control module provided on a second printed circuit board of the handpiece outputs a target high frequency signal preset in association with a temperature, a resistance, and a conductivity of the skin, based on the acquired temperature, resistance, and conductivity of the skin.

10. The high frequency aesthetic medical device of claim 1, wherein the handpiece includes:
a touchscreen panel configured such that an output form of a high frequency signal set to irradiate a high frequency to the skin, a form of a frequency band, and a treatment time are changed through a user interface.

11. The high frequency aesthetic medical device of claim 1, wherein the handpiece is configured to be fastened to the needle tip based on a non-contact magnetic coupling.

12. The high frequency aesthetic medical device of claim 1, wherein a fastening strength of the needle tip and the handpiece is automatically adjusted depending on a condition of the skin.

13. The high frequency aesthetic medical device of claim 1, wherein the needle tip is provided with single-crystal sapphire, and
wherein the needle tip includes a PTFE insulating coating layer formed on an outer surface of the needle tip and configured to apply high frequency energy to the skin through a capacitive coupling scheme between a plurality of electrodes and the skin.

13. The high frequency aesthetic medical device of claim 1, wherein the handpiece includes:
a feedback module configured to measure an impedance of the skin by using a sampling frequency, and to compensate a high frequency output such that a measured impedance value is maintained within a preset target impedance range.

15. The high frequency aesthetic medical device of claim 1, wherein the handpiece includes:
a contact cooling module configured to cool an epidermis by spraying dynamic cooling device (DCD) gas before or after a high frequency is irradiated to the skin.

16. The high frequency aesthetic medical device of claim 15, wherein the handpiece includes:
a drug delivery module configured to spray a drug to the skin in synchronization with an irradiation of the high frequency.

17. The high frequency aesthetic medical device of claim 6, wherein a high frequency generating module provided in the second printed circuit board includes:
a phase synchronization circuit configured to selectively or simultaneously output at least one high frequency signal, and to maintain a phase error between the output high frequency signals at a preset level or less.

18. The high frequency aesthetic medical device of claim 17, wherein the phase synchronization circuit simultaneously applies a plurality of high frequency signals or performs sequential switching based on a direct digital synthesis (DDS) scheme.

19. The high frequency aesthetic medical device of claim 4, wherein the high frequency energy is selected as a single frequency or as multiple frequencies, and
wherein a processor performs a control such that an output, a phase, and a frequency of the high frequency energy are set mutually differently.

20. A method for controlling a high frequency aesthetic medical device, the method comprising:
preliminarily cooling skin by performing contact cooling on the skin through a needle tip, by a processor of the high frequency aesthetic medical device;
monitoring an impedance of the skin in real time while a plurality of electrodes are driven at a preset period to be moved forward to a target depth, by the processor;
phase-synchronizing at least one high frequency energy through a phase synchronization circuit and applying the high frequency energy, by the processor;
measuring the impedance by using a sampling frequency, and automatically adjusting a high frequency output in response to a measured impedance value, by the processor; and
spraying a drug to the skin through a drug delivery module such that the spraying is synchronized with application of a high frequency energy, by the processor.
